(19) United States
(12) Patent Application Publication
(10) Pub. No.: US 2011/0129891 A1
(43) Pub. Date: Jun. 2, 2011

(54) Process for Controlled Homogeneous Acid Leaching

(76) Inventor: Chris du Plessis, Western (AU)

(21) Appl. No.: 13/000,494

(22) PCT Filed: Jul. 29, 2009
(86) PCT No.: PCT/AU09/00961

§ 371 Date: Feb. 17, 2011

(30) Foreign Application Priority Data
Jul. 29, 2008 (AU) 2008903868

(51) Int. Cl. C12P 3/00; C22B 5/00

(52) U.S. Cl. 435/168; 75/375; 75/384

(57) ABSTRACT

A method for leaching a material containing one or more target metals using an acidic leaching solution to extract said one or more target metals, said method including (I) empirically determining an optimal acid concentration range for said acidic leaching solution by: (a) determining the relationship between the concentration of extracted target metal/s and acid consumption in said leaching solution, (b) utilizing said relationship to evaluate value parameters for the target metal containing material as a function of said acid consumption, and (c) determining said optimal acid concentration range, which is the pH range corresponding to an optimal value parameter; and (II) controlling the concentration of said acidic leaching solution such that its pH is substantially within the optimal acid concentration range throughout said material.

### FIELD OF THE INVENTION

This invention relates to a process for acid leaching of a material containing one or more target metals in which the acid concentration of the leach solution is controllable at a level predetermined to be economically optimal. The invention particularly relates to a process for heap leaching of a highly acid consuming material by controlling the acid concentration of the leach solution at an economically optimal and substantially homogeneous level throughout the heap.

### BACKGROUND OF THE INVENTION

Heap leaching is a well-known hydrometallurgical methodology typically used to leach metals from low-grade ores or ore rejects.

Highly acid consuming ores are ores where the target metals require acidic solutions in order to be extracted and the gangue mineralogy also consumes acid at a high rate.

Heap leaching is generally not used for highly acid-consuming ores due to the high acid consumption and resulting impact on: (1) acid-related extractive hydrometallurgy costs, (2) downstream processing costs related to acid-solubilized non-valuable metals, and (3) heap instability.

The notable exceptions are where the extractable metal value is relatively high. Nickel laterites may have relatively high nickel grades (e.g. >0.5%) and the presence of cobalt which have been sufficient to induce application of acid-based heap leaching to nickel laterites, despite the technical and economic impact of high acid consumption.

The main factors that determine whether the heap leaching of a particular ore is economical are:

(1) the overall acid consumption, which is a function of the gangue mineralogy and the acid concentration the ore is exposed to,

(2) the extent of valuable metal extraction, and

(3) the extent of co-extraction of unwanted elements, which impact on downstream processing costs. For example, in the case of nickel laterites both nickel and cobalt are valuable metals released during acid leaching, whereas iron, manganese, aluminium, chromium, and magnesium are the main co-extracted factors that impact on the downstream processing costs.

Acid consumption is the most important deterministic factor of both operating expenditure and capital expenditure in any heap leaching project, because of the above factors.

For a given ore mineralogy the acid consumption, and its associated impact, is governed by the acid concentration at which the leaching process is operated.

Heap leaching of highly acid consuming ores, results in a steep acid concentration gradient as a function of heap height, when operated under typical conditions (i.e. application of acid via the irrigation solution to the top of the heap). This typical mode of heap operation means that the top portion of the heap is overexposed to acid in order to ensure that a minimum concentration of acid eventually reaches the entire bed depth. Such gradient-inducing acid leaching results in an overall unnecessarily high acid consumption and high level of co-dissolution of unwanted metals. This in turn results in the need for a large and expensive acid production plant in addition to the high costs associated with removal of the unwanted metals in the downstream processing circuit.

The high acid consumption and gangue dissolution also often result in heap stability and hydraulic permeability problems. This is because the overall mass loss for typical laterite heap leaching may be as high as 40%, by weight.

One potential way in which the steep pH gradient can be reduced is to increase the irrigation flow rate. This strategy is, however, limited by the fact that severe hydraulic problems are encountered at high solution application rates (>40 L m⁻²h⁻¹) that would be required to overcome the gradient.

Other potential strategies to reduce the gradient include the use of either very low (i.e. <4 m height) heaps or heaps irrigated at multiple levels (i.e. at various depths in addition to the surface irrigation). Both these strategies are also problematic. Very low heaps mean that very large heap surface area and extended heap containment pad footprints are required. This has a significant impact on the capital and operational costs. Multi-layer (or multi-level) irrigation systems, in turn, are impractical due the fact that such irrigation distribution systems are prone to damage during stacking, and cannot satisfactorily be monitored or maintained during heap operation. Such multi-layer irrigation also results in increasing hydraulic flow rates with increasing heap depth, which may have secondary negative effects.

Highly acid consuming ores may also be subjected to pre-treatment steps prior to heap leaching, such as acid treatment, in order to at least partially neutralise the ore. However, such pre-treatment significantly adds to the cost and complexity of treating the ore.

The use of in situ acid generation within the heap addresses some of the limitations listed above. However, to date there has been no rigorous method developed to determine and maintain the optimal leaching conditions in the heap in order to minimise acid consumption but allow sufficient target metal dissolution.

There is accordingly a need for a heap leaching process which avoids acid concentration gradients within the heap and enables leaching to be conducted at a selected acid concentration which can be maintained substantially uniformly throughout the heap. Such a process would allow for leaching to be conducted under economically optimal conditions, taking into consideration valuable metal recovery, acid consumption and downstream processing costs.

### DESCRIPTION OF THE INVENTION

According to the present invention, there is provided: a method for leaching a material containing one or more target metals using an acidic leaching solution to extract said one or more target metals, said method including

(I) empirically determining an optimal acid concentration range for said acidic leaching solution by:

(a) determining the relationship between the concentration of extracted target metal/s and acid consumption in said leaching solution,

(b) utilizing said relationship to evaluate value parameters for the target metal containing material as a function of said acid consumption. and

(c) determining said optimal acid concentration range, which is the pH range corresponding to an optimal value parameter; and

(II) controlling the concentration of said acidic leaching solution such that its pH is substantially within the optimal acid concentration range throughout said material.

The term “value parameter” means a parameter which measures the overall economic value of the metal containing material, taking into account predetermined cost factors. Typically, the value parameter is the net present value, or NPV, of a project involving the heap leaching of the metal containing material. The term “net present value” (NPV) is a term of the art and would be understood by the skilled addressee.

The method of the present invention is particularly applicable to heap leaching a material, and the following description will accordingly focus on this application. However, it is to be understood that the invention is not limited to heap leaching and may, for example, extend to tank leaching, e.g. agitated tanks, or leaching in other vessels.

Accordingly, the leaching process is operated under conditions such that the leaching solution has a substantially uniformly controlled acid concentration that is predetermined to be economically optimal for the particular material being leached.

The target metal may be one or more of cobalt, nickel, copper, zinc and uranium.

Typically the material containing the one or more target metals is selected from ores, concentrates and metal containing waste, and combinations thereof. More typically, the material is an ore, and the following description will focus on this application, although it is to be clearly understood that the invention is also applicable to materials other than ore. More typically, the material is an acid consuming ore, such as a laterite or sulfide-containing ore, preferably a nickel and cobalt containing ore. The nickel and cobalt containing ore may be one or more of laterite, saprolite, nontronite, limonite, partially oxidised and sulfidic ores or a concentrate or intermediate.

Preferably, the target containing material is formed into agglomerates.

Preferably the acidic leaching solution is sulfuric acid.

It is a preferred feature of the present invention that the acidic leaching solution is generated in situ in the heap. While the following description will focus on this embodiment, it is to be clearly understood that the invention is not limited to that embodiment and may extend to conventional methods of application of leaching solutions, such as by addition to the top of the heap or irrigation within the heap

The acidic leaching solution may preferably be generated in situ by either microbial oxidation of a sulfur containing material, or by introduction of a gas mixture comprising SO₂ and an oxygen containing gas. However, due to the potential for fugitive SO₂ emissions, preferably the acidic leaching solution is generated in situ in the heap by microbial oxidation of a sulfur containing material.

The microbial oxidation is preferably effected by sulfur selective micro-organisms which are selected from oxidizing bacteria that are capable of oxidizing sulfur. Non-limiting examples of suitable bacteria or archaea include those selected from the group consisting of Thiobacillus thiooxidans, Thiobacillus ferroxidans, Leptospirillum species, Sulfobacillus, Thermosulfidooxidans, Sulfolobus brierleyi, Sulfolobus acidocaldarius, Sulfolobus BC, Sulfolobus solfataricus, Sulfolobus metallicus, Thiomicrospora sp., Achromatium sp., Macromonas sp., Thiobacterium sp., Thiospora sp., Thiovulum sp., Acidithiobacillus, Acidimicrobium, Sulfobacillus; Ferrimicrobium, Acidiphilum, Alicyclobacillus. Acidianus, Metallosphaera, Thermoplasma and mixtures thereof.

The sulfur selective micro-organisms may include halotolerant microorganisms, such as Thiobacillus prosperus sp nov.

The sulfur containing material is typically selected from elemental sulfur, sulfide compounds and combinations thereof.

The sulfide compounds may include pyrite and pyrrhotite, which may advantageously be abundantly available at some mine sites. The elemental sulfur may comprise relatively coarse sulfur particles, flakes or prills. Coarse sulfur particles, flakes or prills can be used to provide a relatively slow and sustained rate of acid generation. In one embodiment, the elemental sulfur is biologically generated. Such sulfur is often highly reactive and may have hydrophilic surface properties, which can be advantageous at the start of the leach when acid consumption of the ore is high. The fact that bio-sulfur naturally contains microbial species capable of sulfur oxidation at neutral pH, means that this form of sulfur provides the additional benefit of being a useful source of microbial inoculum, again, particularly during the early leaching phase when the solution pH would be relatively high

Preferably, the target metal containing material is formed into agglomerates which include a sulfur containing compound. The agglomerates may additionally include a sulfur-selective microorganism. The sulfur containing compound and sulfur selective microorganism are applied to said metal containing material prior to or during agglomeration. Alternatively, the sulfur containing compound and sulfur selective microorganism may be applied to said metal containing material after agglomeration.

As previously noted, the term “net present value”, or NPV, is a term well-known in the art and refers to the financial appraisal of long-term projects. It is widely used as an economics tool for project evaluation.

A typical formula for calculating NPV is:

$$NPV = - {C}_{0} + {\sum }_{t = 1}^{N} \frac{{C}_{t}}{{( 1 + r )}^{t}}$$

where:

t—the time of the cash flow

N—the total time of the project

r—the discount rate (the rate of return that could be earned on an investment in the financial markets with similar risk.)

Cₜ—the net cash flow (the amount of cash) at time t.

In the case of a project involving heap leaching of an ore, NPV takes into consideration the weighted average cost of capital, financial risk premium, taxation regime, operational costs, capital costs, ore reserve to be treated and a forecast of future metal price. The NPV therefore combines all of the relevant project and company-specific factors to calculate the optimal economic benefit as a function of the most important variable for heap leaching of high acid consuming ores, i.e. acid consumption. The important role of acid consumption is because of the dominant impact of acid requirement and its associated technical impacts on both the capital and operational cost structure of such projects.

In the process of the present invention, step (I) (a) typically comprises:

(i) leaching said material with one or more leaching solutions having a range of pH values to produce one or more leachates

(ii) measuring the concentration of extracted target metal and the acid consumption in the or each leachate;

(iii) determining the relationship between acid consumption and concentration of extracted target metal over the range of pH values.

Preferably, experimental tests are conducted in which the material (usually ore) of interest is subjected to a controlled and constantly maintained pH solution. Exposure to this solution results in the dissolution of the metal containing material, and thus the extraction of the target metal, together with any co-extracted elements, with consequent acid consumption over the selected leaching period at selected optimal particle size. Multiples of these tests, each conducted at a different controlled pH (or acid concentration) level provides overall extraction data for the target metal (and any co-extracted elements) as a function of overall acid consumption, in order to enable determination of the relationship between them. The variation of pH may occur by treatment with a number of leaching solutions, each with a different controlled pH. Alternatively, pH variation may occur in a single leaching solution in which the pH is progressively lowered.

Step (I)(b) utilises the relationship between target metal concentration and acid consumption to evaluate value parameters. This is typically done using the above experimental data, and with knowledge of downstream processing as well as acid generation facilities and their respective project cost implications, determining the net present value (NPV) of a heap leaching project. The NPV can thus be calculated as a function of acid consumption and extraction of the target metal and any co-extracted elements for a specific project.

Step (I) (c) determines the optimal solution pH range for leaching corresponding to an optimal value parameter, which is typically a maximum NPV.

A maximum NPV can be determined for a specific project and its metallurgical extraction behaviour. From the set of experimental tests described earlier, it is possible to determine the (constantly applied) acid concentration, or pH, which resulted in an acid consumption corresponding to the maximum NPV. It should be noted that the maximum NPV does not necessarily coincide with the point of maximum target metal extraction, as will be subsequently discussed.

The acid consumption rate for a given ore is governed mainly by the prevailing solution pH it is exposed to, generally increasing at lower pH levels as depicted in FIG. 1. By contrast acid generation rates via microbial oxidation of sulfur in the preferred embodiment of the invention are very differently affected by prevailing solution pH conditions. In general the sulfur oxidation rate at neutral pH is relatively slow, then increases to a maximum rate at moderately acidic pHs (typically 1.5-3) then rapidly declines again with higher acidity (e.g. pH below 1). This is a generalization and the optimum acid generation range is affected by the specific microbial species comprising the microbial consortium. The conceptual acid generation rate and acid consumption rate are illustrated in FIG. 2. It is important to note that with sufficient sulfur available for oxidation, and sufficient microbial activity, acid generation rates can be made to exceed acid consumption rates at solution pH levels above the equilibrium point. The solution pH will therefore continue to decrease until it reaches the pH equilibrium point. At this point the acid generation rate is equal to the acid consumption rate and no immediate change in solution pH occurs.

The attainment of such an equilibrium condition is firstly dependent upon the ore's acid consumption properties, which in turn are dependent upon both the passage of leaching time and the chemical composition of the leaching solution. The equilibrium condition is secondly dependent upon the acid generation rate and the factors that influence it. Usually, acid generation rate, in this scenario, is more controllable than acid consumption rate properties, and is therefore the main lever used to optimize the leaching conditions. In the case of acid generation by microbial oxidation of sulfur, acid generation is mainly dependent upon:
Sufficient sulfur availability for microbial oxidation.
The physical characteristics of sulfur. Coarse sulfur can be used to provide a relatively slow rate of acid generation. Alternatively, biosulfur typically provides high acid generation.
Sufficient microbial activity and supplementary conditions that contribute to the growth rate of sulfur oxidizing bacteria, including oxygen, carbon dioxide, and other nutritional requirements. Suitable sulfur oxidation microbial species, both bacterial and archaeal, may be added either during agglomeration, ore stacking, as an aerosol after stacking, or via the irrigation solution, as is known in the art.
The microbial species present or introduced. Different microbial species are active at different optimal pHs. For example some bacteria and archaea have a pH optimum between 0 and 1.
Microbial sulfur oxidation activity is also influenced by the presence or absence of inhibitory substances or buffering agents.

From FIG. 2 it can be illustrated that acid generation, particularly at the exponential phase of microbial growth should preferably be curtailed, if the prevailing pH of the solution is lowered below the optimal pH for leaching. An excessively high acid generation rate may have several negative impacts. Firstly it may result in an excessively low solution pH which may result in the negative impacts highlighted earlier. Secondly, an excessively high initial acid generation rate may leave too little remaining sulfur in order to sustain the targeted equilibrium pH condition for the entire leaching period.

The acid consumption rate curve in FIG. 2 represents early conditions where the gangue minerals are at their most reactive. However, with continued acid leaching, the reactivity and thus acid consumption rate of the ore will inevitably decline. With a declining acid consumption rate, preferably the acid generation rate should also be reduced in order prevent the solution pH dropping too low.

With successful controlled homogenous acid leaching achieved the leaching at the targeted pH equilibrium point in the manner described above, the metal extraction can proceed and provide the optimal economic project benefit.

In step (II) of the process of the invention, the concentration of the acidic leaching solution is controlled such that its pH is substantially within the optimal acid concentration range. Typically the concentration of said acidic leaching solution is controlled by controlling the in situ generation of acid within the heap and/or by use of a pH buffering agent. Preferably, step (II) comprises controlling a substantially homogeneous concentration of acidic leaching solution throughout the height of the heap.

Where the acidic leaching solution is sulfuric acid which has been generated in situ, preferably, the in situ generation of sulfuric acid is controlled by one or more of the following mechanisms:

(i) controlling the oxidation of sulfur by regulating the distribution rate of an oxidant within the heap;

(ii) controlling concentration of salts inherently produced during said leach

(iii) controlling the irrigation flow rate within the heap; and

(iv) addition of a buffering agent with a pKₐ value within the selected target pH range.

In the case of mechanism (i), the oxidation of sulfur may be controlled by regulating the flow rate of an oxidising gas throughout the heap. Typically, the oxidising gas is air. The flow rate adjustment may occur in response to measured values of said oxidizing gas within said heap and/or pH of said leaching solution. Typically the heap is aerated via an air distribution system within or under the heap. Because the oxidation of sulfur is an oxygen-consuming process, the restriction of air flow rate has an immediate and readily controllable impact on acid generation. The flow rate of air through the line may be adjusted as necessary. Measurement of oxygen gas concentrations within the heap can be used in addition to solution pH measurements in order to monitor the effect of restricted air flow.

In the case of mechanism (ii), several species may naturally build up within the leaching circuit and may be allowed to reach a level that becomes inhibitory to microbial sulfur oxidation. The inhibitory salts preferably include one or more of magnesium, aluminium, iron, sulfates and chlorides.

The most prevalent of these inhibitory salts in this context is sulfate. Sulfate is particularly useful in this regard because it is not toxic or directly inhibitory per se but rather, causes gradual inhibition because of osmotic and water activity effects. Where the inhibitory salts are sulfates, preferably step (ii) comprises regulating the concentration of sulfate to within the range of 100 to 180 g/L. These inhibition ranges are dependent on the composition of the balancing cations, with monovalent cations (such as sodium) generally causing slightly increased inhibition compared to divalent cations (such as magnesium). The concentration of inhibitory salts can be regulated by managing the leaching solution chemistry and recycle parameters, such as by controlling the amount of fresh leaching solution added to the leachate as it is recirculated within the heap.

In the case of heap leaching a nickel and cobalt containing ore, the inhibitory salts typically comprise magnesium and/or iron sulfates which naturally build up within the leaching circuit, or can be derived from downstream barren leachates. For example the concentration of Mg in solution can be used to control the rate of S oxidation, and therefore the acid generation rate. Mg concentrations, typically with sulfate as the main counter-anion starts becoming inhibitory to S oxidation at soluble concentrations above about 15 g/L. This inhibition is gradual and may only reach its full inhibitory effect at about 30 g/L. This provides a very useful control mechanism for sulfur oxidation. The concentration of Mg in solution can be controlled by the management of solution recycle and the dilution with fresh water. In addition, it may be controlled by membrane filtration or reverse osmosis techniques.

Other inhibitory salts which may be used in a similar manner include chlorides, although chlorides have a more severe inhibitory impact and at much lower concentrations than that of sulfates, and may therefore be more difficult to use as a control mechanism in some embodiments. The use of chlorides may also be complicated by the fact that halo-tolerant bacteria can tolerate an order of magnitude higher chloride concentration than commonly used sulfur oxidation microbial strains. In addition, in some instances such chloride-resistant strains may be deliberately introduced as a major constituent of the microbial inoculation consortium in cases where high-chloride process water is utilized, such as sea water or hypersaline water. Several halotolerant bacteria can also oxidise ferrous iron, resulting in its precipitation within the heap. This advantageously can reduce the amount of iron needing to be processed downstream. In chloride rich process solutions, ferric iron may precipitate as the mineral akaganeit [FeO(OH,Cl)]. Precipitation of this mineral may also be used as a mechanism to remove chlorides from solution, in scenarios where this may be desirable.

Another benefit derived from the use of chloride rich solutions (from concentrations containing 10 g L⁻¹) is that the chloride influences the surface properties of elemental sulfur. More specifically the hydrophobicity of typical Claus-sulfur seems to be reduced, rendering the sulfur more reactive and more readily oxidized by microbial means. This may be used as a means of overcoming prolonged lag time and slow oxidation rates of sulfur where it may occur.

Chlorides also generally have a beneficial effect on leaching performance of minerals, and is generally known in the art. The beneficial effect of chloride is believed to be partly due to the impact of increased proton activity, and because it acts as a complexing agent for iron.

In the case of mechanism (iii), control of the irrigation flow rate may be used either by itself or together with control of aeration flow rate to manage the temperature of the heap, which in turn affects microbial activity. Increasing the flow rate of one or both reduces heap temperature. Irrigation flow rate also has an impact on the prevailing salt content within the heap and may be used as a control mechanism in this manner too. The irrigation flow rate typically is controlled to within the range 6-20 L m⁻²h⁻¹. By increasing the temperature of the heap, generally the rate of leaching also increases. In addition, higher temperatures result in a higher amount of iron precipitates in the heap which can advantageously minimise the amount of iron which needs to be removed in downstream processing.

In the case of mechanism (iv) the buffering agent preferably controls the pH solution either by itself, or in conjunction with another mechanism. pH buffers can typically buffer against mild fluctuations of pH. Accordingly where the acid demand is low, buffers may be able to be used on their own without the need for in-situ acid generation. This would apply in low-acid consuming scenarios or where use of large amounts of buffers is economical. Alternatively, pH buffers can be used in conjunction with another mechanism in high acid consuming environments.

Examples of buffering agents which may be used are oxalate/oxalic acid, phosphate containing species, or any other suitable buffer with a pKₐ values within the target pH range for leaching.

The invention will be better understood by reference to the accompanying drawings and non-limiting Example.

### BRIEF DESCRIPTION OF THE DRAWINGS

FIG. 1 is a graph showing the general relationship between overall acid consumption of an ore and pH of a leaching solution.

FIG. 2 is a graph showing the conceptual acid generation and acid consumption rates as a function of pH, and where the acid generation is biogenically generated from the oxidation of sulfur.

FIG. 3 is a graph showing metal extraction (%) vs acid consumption (kg/ton ore) for a nickel and cobalt laterite ore as described in the Example.

FIG. 4 is the same graph of FIG. 3 additionally showing the net present value vs acid consumption.

FIG. 5 is a graph showing the acid consumption plotted as a function of pH for a nickel and cobalt containing ore described in the Example.

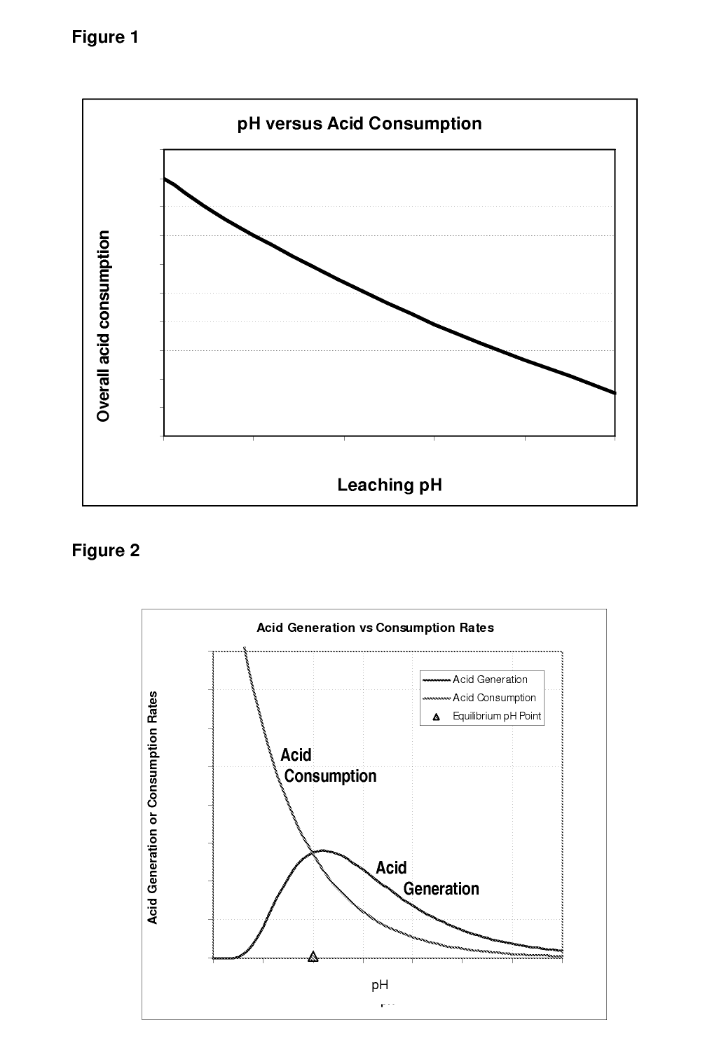

### EXAMPLE

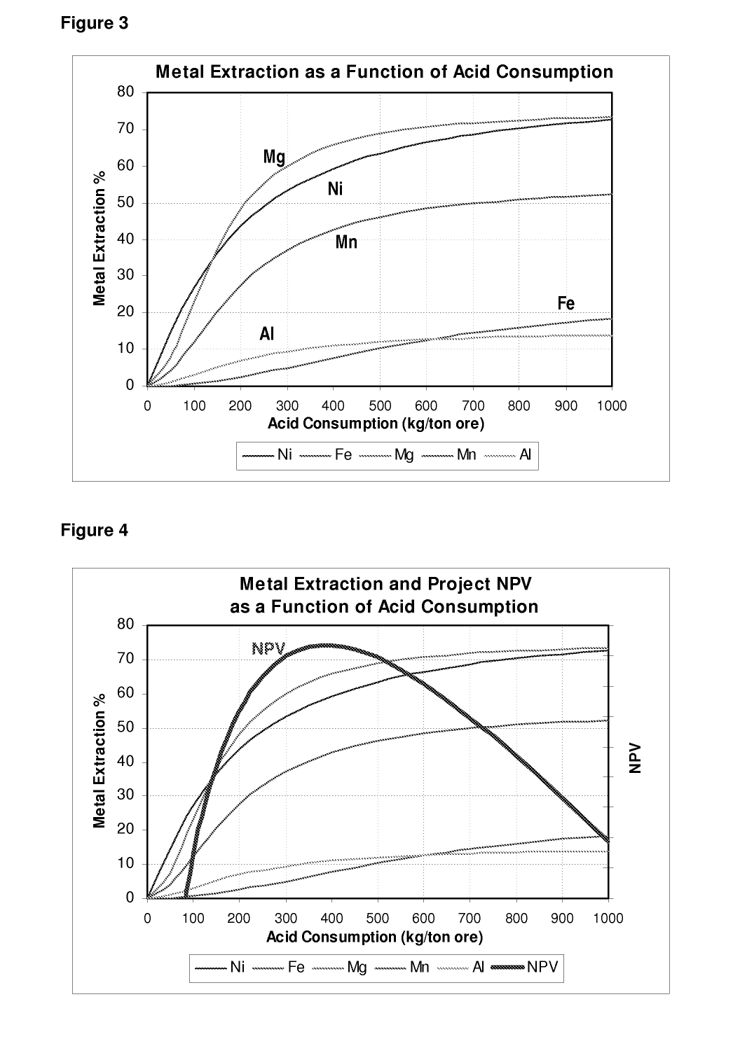

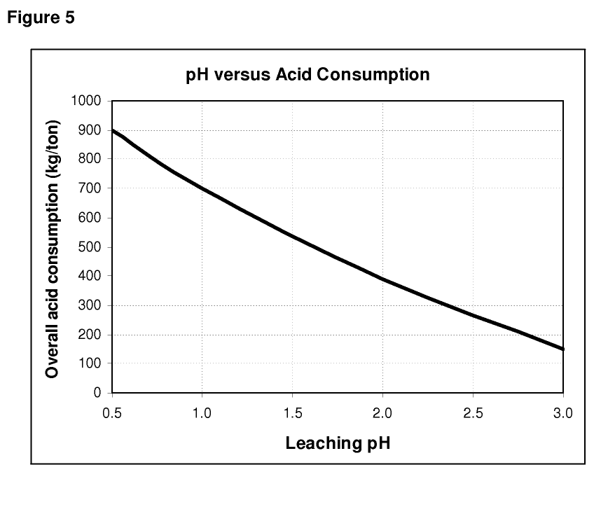

The Example concerns a project involving heap leaching of a lateritic ore using in situ generation of acid. The nickel and cobalt containing lateritic ore of interest is first subjected to experimental leaching using a multiple of leaching solutions, each having a different constant pH. Leaching resulted in the extraction of the metals including nickel, iron, magnesium, manganese and aluminium. The amount of metal extracted by each leaching solution was measured as a function of pH value. Also the acid consumption was plotted as a function of pH in FIG. 5. The percent metal extraction for each metal was then plotted against acid consumption in FIG. 3.

The acid consumption data, together with other project parameters including the weighted average cost of capital, financial risk premium, taxation regime, operational costs, capital costs, ore reserve and a forecast of future reagent and metal prices, were then used to calculate values of net present value of the project. The NPV values were then plotted against acid consumption in FIG. 4.

The scale values for the NPV curve in FIG. 4 are not shown as they are dependent on the long-term price protocols used by the specific company contemplating the project. The shape of the curve, and the principle represented thereby are of greater relevance than specific NPV values per se.

Using FIG. 4 it can be illustrated that the optimal economic acid consumption level to pursue does not necessarily correspond with the maximum extractable target metal of interest. It is clear from FIG. 4 that the maximum NPV is reached for the specific project, at a nickel extraction of less than 60%, despite the fact that the maximum extractable nickel may be over 70%. Without the data collected in the experimental manner as described above, and subsequent techno-economic modelling, it would not be obvious which operating solution pH to target using in-situ acid generation techniques.

Accordingly, FIG. 4 shows that the optimal NPV for the project corresponds to an overall acid consumption of approximately 380kg/ton of ore. The specific acid consumption in turn was the result of a constantly applied pH of leaching solution of approximately 2, as is evident from FIG. 5. Accordingly, the optimal operational heap leaching solution for this project was at pH=2.

Having experimentally determined the optimal solution pH, this knowledge can then be applied to a specific heap leaching project. However, it should be appreciated that knowledge of the optimal solution pH is only of value if the pH within the heap can be controlled at this value. Simply adding a leaching solution having a pH of 2 to the top of the heap is not sufficient because pH will rapidly increase as the solution percolates down the heap.

The laterite ore (have a particle size of 100% passing 6 mm or 12.5 mm) would typically be mixed with sulfur at a concentration of 100-130 kg sulfur per ton during agglomeration. Sulfur oxidising microbes would typically be also added during agglomeration. These may be supplemented, if necessary, by subsequent further additions to the irrigating leach solution. The heap should also include an aeration distribution system. The air flow rate can then be modified in response to pH readings.

The pH levels would typically be determined by measuring the pH of the pregnant leach solution exiting the heap, checking it against the pH of the irrigating leach solution entering the heap and adjusting pH as necessary.

When pH levels drop below the optimal value of 2, air flow rate can be decreased and when pH levels exceed 2, air flow rate can be increased.

By controlling the pH within the heap in this manner, acid consumption can be maintained around the optimal rate of 400 kg/ton, allowing nickel to be extracted at around 60 wt%.

The process of the present invention is not applicable to all ore types. For example, if the ore's acid consumption rate is too high it would prevent a suitably low equilibrium pH condition to be reached using the inventive process. Alternatively if the solution pH required for optimal metal recovery is too low (i.e. <<pH 1), it may not be achievable by microbial oxidation of sulfur. The overall acid consumption is also important, e.g. for a specific ore the leaching time required may be so long that the overall acid consumption is too high to be met by the amount of pre-agglomerated sulfur. Typically, if the overall acid consumption required exceeds 450 kg per ton it would be unlikely that the inventive process, alone, could be successfully applied to the specific ore. This is because an excess of 450 kg sulfur acid would require an agglomerated sulfur content exceeding 150 kg per ton ore, and because exceeding this amount of agglomerated sulfur may generally be problematic for agglomeration and heap stability reasons.

The main advantages of the inventive process are:
Reduced overall acid consumption due to the sustained controlled pH maintained throughout the heap height at the economic optimal leaching pH condition, has an important impact on the project operational costs—both for the extractive hydrometallurgy and downstream processing components of a heap leaching project.
The in situ generation of acid within the heap, allows the significant capital cost of an acid-generation plant to be omitted from the heap leaching project costs.
The less aggressive acid leaching conditions, compared to that demonstrated by traditional acid heap leaching reduces overall mass loss in the heap despite the fact that sulfur mass loss obviously occurs. The reduced mass loss, in turn, reduces the risk of heap instability and hydraulic permeability problems. In addition less aggressive agglomeration techniques can be used—allowing significantly reduced acid requirements during agglomeration. Acid requirement during agglomeration has to be determined on a case-by-case basis but stable agglomeration required for the process of the invention has been demonstrated with the use of acid additions as low as 2-5 kg per ton of ore during agglomeration.
Increased heap stability facilitated by the invention, allow for taller heaps to be used compared to the typical 4-5 metre height used for highly acid consuming ores. Increased heap heights may significantly reduce the heap footprint, leaching pad, and thus also the ancillary capital cost items such as irrigation, aeration and drainage systems.
The conditions conducive to microbial sulfur oxidation are generally also conducive to ferrous iron oxidation. In addition many of the microbial strains capable of sulfur oxidation also have the ability to oxidize ferrous iron. Iron oxidation within a heap is an important benefit for a number of reasons. Several target heap leaching projects result in significant release of iron in the ferrous state. Iron, and ferrous iron in particular, poses a significant cost impact on downstream processing, most notably in the case of laterite projects. The oxidation of ferrous iron within the heap improves the extent to which iron precipitates and is retained within the heap. Such precipitation generally occurs as jarosite, schwertmannite or other ferric oxyhydroxides. This reduces the amount of iron that reports to the leach solution and downstream processing circuit. In addition, ferric sulfates which may also be produced in the heap are inhibitory salts which can control pH in the heap as previously discussed.
Another, important benefit of iron precipitation in this manner is that it contributes additional acid into solution, as a result supplementing the acid produced from sulfur oxidation. The precipitation of jarosite may be induced by the presence of cations such as sodium. The general reaction for jarosite (and related precipitates) is given below:

A⁺+3Fe³⁺+2SO₄²⁻+6H₂O→AFe₃(SO₄)₂(OH)₆+6H⁺

(where A represents cations such as K⁺, Na⁺, NH₄⁺, or H₃O⁺)
Elevated heap temperatures may be attained within the heap, depending upon the imposed heat loss strategy employed by controlling the combination of air flow rate, irrigation rate and heap height. A significant amount of heat may be available from the oxidation of sulfur (i.e. S⁰+1.50₂+H₂O→SO₄²⁻+2H⁺ yields ΔH⁰ⱼ=−606 kJ mol⁻¹). Increasing temperature may in turn increase heap leaching kinetics and reduce leaching periods. Anticipated increased heap temperatures may require the inoculation of the heap with microbial strains suitable for elevated temperature conditions.
Increased temperatures also significantly increase precipitation of jarosite, schwertmannite and similar minerals, thereby further increasing acid derived from this source as well as benefiting downstream processing.
Iron precipitation as described here also results in reduced sulfate reporting to the downstream processing circuit. This is an important benefit in high rainfall environments where sulfate treatment and/or disposal cannot be achieved by evaporation methods.
Increased temperature of the pregnant leach solution exiting the heap also results in a reduction in energy requirement for downstream processing. For example, a typical process used for the removal of iron from pregnant leach solutions is the so-called goethite precipitation process in which ferric iron is precipitated as goethite. For this to occur the solution temperature is typically increased to approximately 70° C. at pH 4.5. This temperature increase typically requires a very large energy consumption, which is reduced if the pregnant leach solution temperature exiting the heap is already at an elevated level.
Arsenic oxidation is often associated with ferrous iron oxidation and is a well-known feature of bioleaching systems, where the arsenic is oxidized to arsenate and co-precipitated with ferric-oxyhydroxides, thus preventing arsenic from reporting to the downstream solution processing circuit. This may be an advantageous feature for applications of low-grade sulphide containing arsenic minerals in high acid consuming ores.

The invention described herein is susceptible to variations, modifications and/or additions other than those specifically described and it is to be understood that the invention includes all such variations, modifications and/or additions which fall within the spirit and scope of the above description.

1-38. (canceled)

39. A method for leaching a material containing one or more target metals using an acidic leaching solution to extract said one or more target metals, said method including:
(I) empirically determining an optimal acid concentration range for said acidic leaching solution by:
(a) determining the relationship between the concentration of extracted target metals and acid consumption in said leaching solution,
(b) utilizing said relationship to evaluate value parameters for the target metal containing material as a function of said acid consumption, and
(c) determining said optimal acid concentration range, which is the pH range corresponding to an optimal value parameter; and
(II) controlling the concentration of said acidic leaching solution such that its pH is substantially within the optimal acid concentration range throughout said material.

40. The method of claim 39, wherein said material is formed into agglomerates and leached by a heap leaching process.

41. The method of claim 39, wherein said metal containing material is selected from one or more of ores, ore rejects, concentrates, metal containing waste, high acid consuming ore, laterite ore, a nickel and cobalt containing laterite ore, low grade sulfide ores, transition ores and supergene ores.

42. The method of claim 39, wherein said metal is selected from nickel, cobalt, copper, zinc and uranium.

43. The method of claim 39, wherein said acidic leaching solution is sulfuric acid and is generated in situ in the heap by microbial oxidation of a sulfur containing material.

44. The method of claim 43, wherein said microbial oxidation is effected by sulfur oxidising micro-organisms selected from the group consisting of Thiobacillus thiooxidans, Thiobacillus ferroxidans, Leptospirillum species, Sulfobacillus, Thermosulfidooxidans, Sulfolobus brierleyi, Sulfolobus acidocaldarius, Sulfolobus BC, Sulfolobus solfataricus, Sulfolobus metallicus, Thiomicrospora sp., Achromatium sp., Macromonas sp., Thiobacterium sp., Thiospora sp., Thiovulum sp., Acidithiobacillus, Acidimicrobium, Sulfobacillus; Ferrimicrobium, Acidiphilum, Alicyclobacillus. Acidianus, Metallosphaera, Thermoplasma halotolerant microorganisms, Thiobacillus prosperus sp.nov and mixtures thereof.

45. The method of claim 43, wherein said sulfur containing material is selected from elemental sulfur, sulfide compounds, pyrite, pyrrhotite, and combinations thereof.

46. The method of claim 45, wherein said elemental sulfur is hydrophilic and includes coarse sulfur particles, flakes or prills.

47. The method of claim 39, wherein the value parameter is net present value (NPV).

48. The method of claim 39, wherein step (I) (a) further comprises:
(i) leaching said material with one or more leaching solutions having a range of pH values to produce one or more leachates;
(ii) measuring the concentration of extracted metal in the or each leachate;
(iii) calculating the acid consumption value corresponding to each pH value;
(iv) determining the concentration of extracted metal for each value of acid consumption; and
(v) determining the relationship between concentration of extracted metal and acid consumption.

49. The method of claim 40, wherein said agglomerates include a sulfur containing compound.

50. The method of claim 49 wherein the agglomerates include an inoculum capable of sulfur oxidation.

51. The method of claim 50, wherein said sulfur containing compound and said microbial inoculum are applied to said metal containing material either prior to, during or after agglomeration.

52. The method of claim 39, wherein said optimal acid concentration range is between pH of about 0.5 and about 2.5.

53. The method of claim 39, wherein in step (II), the concentration of said acidic leaching solution is controlled by controlling the in situ generation of acid within the heap.

54. The method of claim 39, wherein step (II) comprises controlling a substantially homogeneous concentration of acidic leaching solution throughout the height of the heap.

55. The method of claim 44, wherein said in situ generation of sulfuric acid is controlled by one or more of the following mechanisms:
(i) controlling the oxidation of sulfur by regulating the distribution rate of an oxidant within the heap;
(ii) controlling concentration of inhibitory salts inherently produced during said leach, such as magnesium, sulfates and chlorides;
(iii) controlling the irrigation flow rate within the heap, preferably within the range of about 6 to about 20 L m⁻²h⁻¹;
(iv) addition of a pH buffering agent.

56. The method of claim 55, wherein mechanism (i) comprises regulating the flow rate of an oxidising gas throughout the heap.

57. The method of claim 55, wherein mechanism (i) comprises adjusting the flow rate of an oxidising gas throughout the heap in response to measured values of said oxidizing gas within said heap and/or pH of said leaching solution.

58. The method of claim 55, wherein mechanism (ii) comprises regulating the concentration of sulfate to within the range of 100 to 180 g/L.

59. The method of claim 55, wherein mechanism (iii) comprises regulating the irrigation flow rate in order to control heap temperature.
